(19) United States
(12) Patent Application Publication
(10) Pub. No.: US 2001/0027250 A1
(43) Pub. Date: Oct. 4, 2001

(54) Activator protein of human acid maltase and uses thereof

(76) Inventor: Frank T. Martiniuk, Wood Ridge, NJ (US)

Correspondence Address: MERCHANT & GOULD PC, 1050 17TH STREET, SUITE 1400, DENVER, CO, 80265, US

(21) Appl. No.: 09/728,997

(22) Filed: Dec. 1, 2000

Related U.S. Application Data
(60) Provisional application No. 60/087,624, filed on Jun. 2, 1998.

(30) Foreign Application Priority Data
May 26, 1997 (US) PCT/US99/11679

(51) Int. Cl. A61K 38/47; C07K 16/40

(52) U.S. Cl. 530/388.26; 424/94.61

(57) ABSTRACT

The present invention relates to compositions and methods for treating glycogen storage disease, such as GSD II. A human activator enzyme, termed ASA, of acid a a-glycosidase, (GAA) or acid maltase, a lysosomal enzyme, is specifically defined and characterized. The AGA has been found to increase the activity of human acid α glycosidase activity to at least 10-fold, relative to the activity of GAA in the absence of the activator protein. The invention thus also provides for a method of increasing the activity of GAA, particularly through the action of AGA. The AGA has an approximate molecular weight of 25-30 kD, and is found to be heat stable. In addition, the AGA is found to have an extended shelf life without significant loss of ability to activate GAA. The invention further reports other enzymes such as β-Fucosidase, β-Lactase, and β-Galactosidase, that provide enhancement of enzymatic activity nine-fold, six-fold, and five-fold, for breakdown of their respective substrate protein. These enzymes are non-lysosomal enzymes. These are anticipated to be useful in treatment of disease related to reduced enzymatic activity levels in an animal.

The present application claims priority to a Provisional U.S. Patent Application Ser. No. 60/087,624, filed Jun. 2, 1998, which was followed by international application PCT/US99/11679, filed May 26, 1997, which was followed by a U.S. application, Serial No. to be assigned, filed Dec. 1, 2000.

The United States Government may own rights to the invention as research was supported by National Institutes of Health Grant #ROI 39669-01.

### FIELD OF THE INVENTION

The present invention relate generally to the fields of therapeutic formulation methods of their preparation. More particularly, it concerns a GAA protein as well as an AGA activator protein, and therefore relates to the field of new proteins, both naturally occurring and as recombinately produced.

### BACKGROUND OF THE INVENTION

Glycogen storage diseases are exemplified by a pathology known as Glycogen Storage Disease type II (GSD II). This is autosomal recessive disorder exists in three forms or types. One type is the fatal infantile form, also known as Pompe's disease, which usually results in death by the first year. It is characterized by general hypotonia with massive accumulation of glycogen in all tissues, including cardiac as well as skeletal muscle.

The second type of glycogen storage disease is a more viable juvenile form that is often fatal by the second year of life. There is progressive skeletal involvement that includes respiratory muscles, and death is due to respiratory failure.

The third type is the adult-onset form of glycogen storage disease that occurs later in life. Glycogen storage is limited to skeletal muscle, and there is a weakening of respiratory muscles. The diaphragm is particularly affected, and death again occurs by respiratory failure with preterminal signs of pulmonary hypertension and cardiac failure.

GSD II in all its forms, as well as other glycogen storage diseases, are caused by the absence of enzymes necessary to metabolize glycogen. The requisite enzymes are lysosomal enzymes necessary to metabolize glycogen. The requisite enzymes are lysosomal enzymes exemplified by acid alpha glucosidase (GAA), or acid maltase. To date, there has been no cure or effective treatment for the glycogen storage diseases and, in particular, GSD II.

It has long been recognized that since these diseases result from a lack of the necessary enzymes, enzyme replacement might be a successful method of treatment. However, treatment by enzyme replacement therapy has not been successful for a variety of reasons. A fundamental difficulty underlying all the efforts thus far is the need to have large quantities of the enzymes. It has been estimated that for successful enzyme replacement therapy of GSD II, 100 to 200 mg of GAA would be needed for an infantile-onset patient and 200 mg to 300 mg for an adult-onset patient. In both situations, these doses would be required every week to two weeks.

Efforts to use purified placental GAA have not been successful since each placenta contains only 5 to 10 milligrams of GAA. Also, there are health and safety concerns, based primarily on possible HIV transmission and other transmitted disease contamination.

Efforts to date to generate the necessary large quantities of human GAA have included recombinant technology. Recombinant human GAA in E.coli was produced, but the resultant protein was not enzymatically active. Yeast expression of human GAA resulted in only partial enzyme activity. GAA expressed in the Spodoptera frugiperdai insect-Bacolovirum system generated a protein that was not functional. Mammalian expression systems have suffered due to their inability to produce large quantities of the recombinant protein.

Radin et al. Biochem J. 845-849, 1989 describe a heat stable protein (AGA) that has the ability to enhance the activities of GAA. However, even with this activator, the recombinant human GAA produced as described above still cannot provide adequate levels of GAA activity as required for enzyme replacement therapy.

### SUMMARY OF THE INVENTION

The present invention overcomes the problems of the prior art and provides GAA in quantities sufficient to maintain enzyme replacement therapy for the treatment of glycogen storage diseases. The present invention provides treatments for glycogen storage diseases by enzyme replacement therapy, cell lines that express enzymes suitable for treatment of glycogen storage diseases, and combinations of such enzymes with activator proteins.

Briefly stated, the present invention comprises enzyme replacement therapy for glycogen storage diseases utilizing recombinant human GAA expressed by novel mammalian cell lines, such cell lines, a combination of such recombinant human GAA and activator protein, and the use of such combinations for enzyme replacement therapy for glycogen storage diseases.

A feature of the invention is a stable mammalian cell line capable of expressing human GAA. In particular cell lines, expression is in an amount that represents at least 5% of the total cellular protein.

A further feature of the invention is the cell line ATCC Designation CRL-12457.

The present invention also provides for an effective method of treatment for glycogen storage diseases, particularly GSD II, by enzyme replacement therapy comprising administering an effective amount of the recombinant human GAA produced by a stable mammalian cell line capable of expressing human GAA.

In some embodiments, the stable mammalian cell line used will produce GAA in an amount that represents at least 5% of the total cellular protein.

In another aspect, the present invention provides for a method of treating glycogen storage diseases, particularly CSD II, by enzyme replacement therapy comprising administering an effective amount of the recombinant human GAA produced by the biologically pure and stable mammalian cell line, ATCC Designation CRL-12457.

A further feature of the invention is a composition for treating glycogen storage diseases, particularly GSD II, comprising a recombinant human GAA expressed by a stable mammalian cell line in which said GAA represents at least 5% of the total cellular protein.

Still another feature of the invention is a composition for treating glycogen storage diseases, particularly GSD II, comprising a recombinant human GAA expressed by a stable mammalian cell line in which said GAA represents at least 5% of the total cellular protein and the human GAA activator protein, AGA.

In another aspect, the invention provides for a composition for treating glycogen storage diseases, particularly GSD II, comprising a recombinant human GAA expressed by the biologically pure and stable mammalian cell line, ATCC Designation CRL-12457.

A feature of the invention is one of the foregoing compositions including the human GAA activator protein, AGA.

In still another aspect of the invention, the method of treating a glycogen storage disease by enzyme replacement therapy comprising administering an effective amount of one of the foregoing compositions is provided.

The following abbreviations are used throughout the description of the present invention:

GAA—acid alpha glucosidase (GAA), or acid maltase.

AGA—a heat stable protein that enhances the activity of GAA.

### DETAILED DESCRIPTION OF THE PREFERRED EMBODIMENT

Following long-standing patent law convention, the terms “a” and “an” mean “one or more” when used in this application, including the claims.

While the instant invention is directed to enzyme replacement for the treatment of glycogen storage diseases, it is especially directed to GSD II and will be particularly described in connection therewith.

An element of the instant invention is the use of recombinant human AGA.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The activity of the recombinant human GAA from a cell line is enhanced by the heat stable protein, AGA. Studies conducted show an at least ten fold enhancement. This is significant since it permits a reduction in the amount of human GAA that needs to be used for enzyme replacement therapy. This greatly reduces, if not eliminates, patient immunological and inflammatory responses to the recombinant enzyme.

AGA can be isolated from various mammalian tissues, cells, and urine. It is within the scope of the present invention to use recombinant by expressed AGA. It will also be evident that the optimum amount of AGA to utilize with the human GAA produced by a cell line, for example, can be determined by routine experimentation. The amounts will vary dependent upon such factors as purity of the AGA, quantity of recombinant human GAA required for dosage of a particular patient, and the age and size of the patient. These same factors apply if the recombinant human GAA is used without the AGA.

While the description of the invention has been directed to the human glycogen storage diseases, it will be understood that the essential elements of the invention are applicable to the related glycogen storage diseases of other mammals, such as cattle, sheep, dogs, cats and the like.

### EXAMPLE 1

### AGA Protein—An Identified Protein That Enhances GAA

Crude human urine was examined to determine its ability to enhance human placental GAA activity. About 5 or 50 uL was added to purified human placental GAA and then assayed for GAA activity with the artificial substrate 4-MU-Glyc at pH 4 (Table 1). The crude human urine increased GAA activity 2.7-fold for 5 uL or 3.9-fold 50 uL of urine. The urine was heated to 100° C. for 10 minutes. Identical results were found. This data indicates that human urine contains a factor that enhances human GAA activity, and this factor as described in the present invention is AGA.

TABLE 1

| | | Relative | |
|---|---|---|---|
| | | Fluorescence | Fold |
| | | Units | Increase |
| Human placental GAA-(1 uL = 0.1 mg) | | 130 | 0 |
| Human placental GAA-(1 uL = 0.1 mg). | | | |
| Plus human urine | 5 uL or | 350 | 2.7 |
| | 50 uL | 510 | 3.9 |
| Human urine | 5 uL or | 4 | 0 |
| | 50 uL | 10 | 0 |

Human urine contains a factor that enhances human GAA activity and is heat stable at 100° C.

### EXAMPLE 2

### Effect of Siliconization of Tubes in Concentrated Urine AGA or Dialyzed Protein

Collection of 700 mL of human urine with sodium azide (0.02%) concentrated to 10 mL under nitrogen with a YM10 membrane was assayed for AGA activity. It was found that this enhancer could be concentrated. However, a loss was observed through the membrane. Other membranes with molecular cut off of 3 and 5 kD, dialyzed human urine against various buffers, found the same affect. Test tubes that are siliconized will not bind AGA.

AGA has a unique structure or shape that does allow it to pass through membranes where it should not. Siliconized test tubes will not bind AGA.

### EXAMPLE 3

### Determination of Native Molecular Weight of AGA

Chromatographed human urine on Sephadex G75 and Sepharose 12 in various buffers and assayed fractions for enhancer activity found that there was a loss in activity. There was a broad peak of enhancer activity with the molecular weight of approximately 25-30 kD. AGA on a 4-20% native polyacrylamide gel is lyophilized peak material and electrophoresed and stained half for protein with Comassie Brilliant Blue dye and the other half cut into 1 cm sections, resuspended in buffer and assayed for enhancer activity approximately 3-5 protein bands confirms human urine AGA has a molecular weight of 25-30 kD and is a protein.

### EXAMPLE 4

### AGA Purification

While many methods may be used to purify AGA from a biological sample, the present example demonstrates an AGA partially purified by salt precipitation. This is a relatively rapid way to concentrate and partially purify the AGA. Human urine precipitated with ammonium sulfate at 40%, 60% and 80% saturation, and it was determined that AGA activity was in the precipitate. AGA is precipitated at 40-60% saturation and that approximately three fold purity can be achieved without a major loss. The other protein bands on native PAGE are still present. AGA can be precipitated at 40=60% ammonium sulfate with some purification.

### EXAMPLE 5

### Stability of ABA at 4° C.

Various preparations of AGA purified from human urine as described in example 4 were maintained at 4° C. This AGA was assayed for AGA activity after up to four months of storage. The AGA activity was found to be totally stable at 4° C., with no appreciable loss in activation potency for GAA activity being detected.

### EXAMPLE 6

### Subunit Size of AGA on SDS-PAGE

Even though the protein is not active in the presence of SDS, various preparations of SDS-PAGE were prepaid in order to determine subunit size. These studies were performed using gels stained with Comassie brilliant blue by standard methods.

### EXAMPLE 7

### Biochemical Characteristics

It has been determined that the AGA is not urea. Urea is the most abundant molecule in urine. The time to enhance the GAA is very short, under 10 minutes, and pH is not important below pH 7.5. By increasing the amount of urine AGA with a constant amount of GAA, an increase in enhance, but not proportional to the amount of AGA added, is observed.

### EXAMPLE 8

### Purification and Biochemical Characterization of Human AGA

Human urine was collected with 0.02% sodium azide, heated to 100° C., and cooled by using the following steps:

1. 60% ammonium sulfate fraction;

2. Sephadex G75 chromatography; and

3. DEAE-Sephacyl ion-exchange chromatography.

Analysis of the above material on SDS-PAGE showed three major bands.

Utilizing gradient native PAGE, cutting bands of 1 cm and testing for activity found an area of AGA activity and analysis on SDSPAGE showed the same banding pattern as previous material.

### EXAMPLE 9

### Generation of Monoclonal Antibodies to AGA

AGA was partially purified through identification of several bands on SDS-PAGE by the various methods known to those of skill in the art. This partially purified AGA preparation may be used to immunize animals and provide spleen tissue that may be used to generate a hybridoma cell line that produces monoclonal antibody having specific binding affinity for AGA. The present example demonstrates the utility of the invention for preparing monoclonal antibodies having an affinity for the AGA protein, where the innoculent is AGA comprised of these purified subunit bands.

Monoclonal antibodies in BALB/c mice were generated with either heat inactivated human urine or the material purified as above by giving four injections-IP in incomplete Freuds adjuvant. These injections were given two weeks apart. Hybridomas that produce monoclonal antibody having a binding affinity for each of the individual proteins in the material, one of which is AGA are produced according to the following protocol:

Two BALB/c mice were immunized with partially purified, human urine-extracted, AGA. These mice produced antibodies to this AGA preparation as detected by Ouchterlony double immunodiffusion plates. Hybridomas are currently being made from the splenocytes of these mice.

A subclass of antibody will be determined. One technique that may be used to accomplish this is BioRad's mouse Type Isotype kit. Monoclonal antibodies to AGA will be expanded, and be attached to Sepharose 4B to be used to purify the AGA to homogeneity. N-terminal amino acids will be determined, and used to generate a probe to obtain the cDNA and sequenced and characterized by standard molecular methods.

### EXAMPLE 10

### AGA Activation Protein

The present example demonstrates the utility of non-lysosomal enzymes for activation of other proteins. Other proteins associated with disease states, include galactosidase, mannosidase, and glucuridase. The genes for the Sphingolipid activator proteins will be obtained for comparison. These compounds share properties that may be used to even further characterize the function of the GAA-AGA protein.

adenosine deaminase

nucleoside phosphorylase

beta galactosidase

alpha galactosidase

beta glucuronidase

alpha and beta mannosidase

lysozyme

alpha and beta fucosidase

neuraminidase

beta glucosidase

iduronidase

arabinosidase

lactosidase

### β-Fucosidase

β-Fucosidase is a non-lysosomal enzyme. The presence of this enzyme is demonstrated by the present inventors to enhance activity up to nine-fold. This enzyme was observed to function at a neutral pH of pH 7. This enzyme was characterized in a urine preparation of it. It is envisioned that the enzyme will be further purified, and in such forms, even greater enhancement activity is expected. It is heat stable.

### β-Lactase

β-Lactase is a non-lysosomal enzyme. The presence of this enzyme is demonstrated by the present inventors to enhance activity up to six-fold. This enzyme was observed to function at a neutral pH, a pH of 7. This enzyme was characterized using a urine preparation of it. It is envisioned that this enzyme will be further purified, and in such forms, even greater enhancement activity is expected.

### β-Galactosidase

β-Galactosidase is a non-lysosomal enzyme. The presence of this enzyme is demonstrated by the present inventors to enhance activity up to five-fold. This enzyme was observed to function at an intermediate pH, pH 5. This enzyme was characterized using a urine preparation of it. It is envisioned that the enzyme will be further purified, and in such forms, even greater enhancement activity it expected.

### References

The following references, to the extent that they provide exemplary procedural or other details supplementary to those set forth herein, are specifically incorporated herein by reference.

1. Martiniuk F. and Hirschhorn R., Amer J Hum Genet 32:497-507, 1980.

2. Martiniuk F, Hirschhorn R and Smith M, Cytogenet Cell Genet 27:168-175, 1980.

3. Martiniuk F. and Hirschhorn R., Biochim Biophys Acta 658:2248-2261, 1981.

4. Martiniuk F, Smith M, Desnick, R J, Astrin K, Mitra J and Hirschhorn R., Cytrogenet Cell Genet 35:110-116, 1983.

5. Hirschhorn R, Martiniuk F, Roegner-Maniscalco V, Ellenbogen A, Perignon J-L and Jenkins T, J Clin Invest 82:1887-1892, 1983.

6. Honig J, Martiniuk F, D'Eustachio P, Zamifirescu C, Desnick R, Hirschhorn K, Hirschhorn L R and Hirschhorn R, Ann Human Genet 48:49-56, 1984.

7. Martiniuk F, Honig J and Hirschhorn R, Arch Biochem Biophys 231:454-460, 1984.

8. Martiniuk F, Ellenbogen A and Hirschhorn R, J Biol Chem 260:1238-1242, 1985.

9. Hirschhorn R, Ellenbogen A and Martiniuk F, J Cell Physiol 123:277-282, 1985.

10. Martiniuk F, Ellenbogen A, Hirschhorn K and Hirschhorn R, Hum Genet 69:103-111, 1985.

11. Martiniuk F, Ellenbogen A and Hirschhorn R, Somat Cell Genet and Mol Biol 12:1-12, 1986.

12. Martiniuk F, Pellicer A and Hirschhorn R, J Biol Chem 260:14351-14354, 1986.

13. Martiniuk F, Mehler M, Pellicer A, Tzall S, LaBadie G, Hobart C, Ellenbogen A and Hirschhorn R, PNAS 83:9641-9644, 1986.

14. Retting W J, Yenamandra A K, Grzeschik K H, Martiniuk F, Horschhorn R, Jennings M T, Chaganti R S K and Old L J, Human Gene Mapping 9:681 (Paris), 1988.

15. Hirschhorn R, Martiniuk F, Tzall S, Mehler M and Pellicer A, Oxford University Press, NY pp. 248-257, 1989.

16. Martiniuk F, Mehler M, Tzall S, Meridith G and Hirschhorn R, DNA 9:85-94, 1990.

17. Haines J L, Ozelius L J, McFarland H, Menon A, Tzall S, Martiniuk F, Horschhom R, Breakefield X O and Gusella J F, Genomics 8:1-6, 1990.

18. Martiniuk F, Mehler M, Tzall S, Meredith G and Hirschhorn R, Am J Hum Genet 47:73-79, 1990.

19. Martiniuk F, Bodkin M, Tzall S and Hirschhorn R, Amer J Hum Genet 47:440 445, 1990.

20. Tzall S, Martiniuk F, Adler A and Hirschhorn R, Nucleic Acids Res 18:1930, 1990.

21. Tzall S, Martiniuk F, Adler A and Hirschhorn R, Nucleic Acids Res 18:1661, 1990.

22. Tzall S, Martiniuk F, Ozelius L, Gusella J and Hirschhorn R, Nucleic Acids Res, 19:1727, 1991.

23. Tzall S, Martiniuk F, Adler A and Hirschhorn R, Nucleic Acids Res 18:1727, 1991.

24. Tzall S, Martiniuk F, Biochem Biophys. Res. Comm, 176:1509-1515, 1991.

25. Martiniuk F, Bodkin M, Tzall S and Hirschhorn R, DNA and Cell Biology 10:283-92, 1991.

26. Zhong, N, Martiniuk F, Tzall S and Hirschoom R, Am J Hum GeneticsU 49:635-645, 1991.

27. Martiniuk F, Hirschhorn R and D'Eustachio P, Mammalian Genome 1:267-269, 1991.

28. Martiniuk F, Mehler, Bodkin M, Tzall S, Hirschhorn K, Zhong N and Hirschhorn R, DNA and Cell Biology 10:681-687, 1991.

29. Martiniuk F and Chen A, DNA and Cell Biology, 11:701-706, 1992.

30. Martiniuk F, Healy P, Howell J M, Tzall S and Hirschhorn R, Vet. Pathology (Accepted) 1993.

31. Martiniuk F, Tzall S and Chen A, DNA and Cell Biology 11:701-706, 1992.

32. Martiniuk F, Mehler M, Tzall S, Meredith G, and Hirschhorn R, DNA and Cell Biology __:85-94, 1992.

33. Martiniuk et al (1998), American Journal of Medical Genetics, Letter to the Editor, 79:69-72.

34. Raben N, Nichols R C, Martiniuk F, and Poltz P H, Human Molecular Genetics 5(7):995-1000, 1996.

We claim:

1. A composition comprising a human activation protein of human acid maltase (AGA), said activation protein being capable of enhancing human placental acid alpha glucosidase (GAA) activity at least 10-fold over GAA activity in the absence of human acid maltase (AGA).

2. The composition of claim 1 when the activator protein is further defined as having a molecular weight of about 25 kD to about 30 kD as determined on a Sephadex G75 column.

3. The composition of claim 1 farther defined as heat stable.

4. A monoclonal antibody having specific binding affinity for a human activation protein of human acid maltase, produced by a process of:
injecting a mammal capable of mounting an immune response upon exposure to an antigen with an immune-response evoking amount of heat inactivated human urine or a composition of claim 1;
repeating the injection four times, two weeks apart;
monitoring sequential blood samples from the injected animal to determine a titer of polyclonal AGA antibody levels in the animal;
collecting the spleen from each treated animal;
preparing a hybridoma cell line from said spleen and an immortalized cell line;
culturing said hybridoma cell line for a period of time suitable for production of monoclonal antibody from said hybridoma cell line; and
collecting monoclonal antibody having specific binding affinity for human activation protein of human acid maltase.

5. The monoclonal antibody of claim 4 wherein the animal is injected with heat inactivated urine comprising a human activator protein (AGA) of human acid maltase.

6. A method for increasing acid alpha glucosidase activity in a patient having acid maltase deficiency comprising administering an effective amount of the human GAA activator protein, AGA of claim 1.

7. The method of claim 6 wherein the acid maltase deficiency is further defined as GSD II.

8. The composition of claim 1 wherein said activator protein is further defined as having a molecular weight of about 25-30 kD.

9. A composition comprising β-Fucosidase, said β-Fucosidase being capable of enhancing biological activity up to nine-fold.

10. A composition comprising β-Lactase, said β-Lactase being capable of enhancing biological activity of up to six-fold.

11. A composition comprising β-Galactosidase, said β-Galactosidase being capable of enhancing biological activity up to five-fold.
